(19) United States
(12) Patent Application Publication
(10) Pub. No.: US 2022/0313239 A1
(43) Pub. Date: Oct. 6, 2022

(54) IMPLANTABLE REPAIR DEVICES

(71) Applicant: HELIX FIXATION, INC., Pleasanton, CA (US)

(72) Inventor: John F. KRUMME, Pleasanton, CA (US)

(21) Appl. No.: 17/640,953

(22) PCT Filed: Sep. 14, 2020
(86) PCT No.: PCT/US2020/050620

§ 371 Date: Mar. 7, 2022

Related U.S. Application Data
(60) Provisional application No. 62/899,474, filed on Sep. 12, 2019.
(60) Provisional application No. 62/970,164, filed on Feb. 4, 2020.

(51) Int. Cl. A61B 17/04; A61B 17/72

(52) U.S. Cl. CPC A61B 17/0401; A61B 17/7225; A61B 2017/0409; A61B 2017/0414; A61B 2017/681

(57) ABSTRACT

An implantable device may be provided with first and second bone anchors, at least one suture and a force-generating component. In some embodiments, the first bone anchor is configured to anchor in or on a first bone segment and the second bone anchor is configured to anchor in or on a second bone segment. The at least one suture may interconnect the first bone anchor and the second bone anchor. The force-generating component may be connected to the at least one suture and configured to impart a force thereto. The first bone anchor, the second bone anchor, the at least one suture and the force-generating component may be configured to cooperate together to draw the first bone segment and the second bone segment toward one another with a force in a predetermined range. Methods of device construction and use are also provided.

### CROSS REFERENCE TO RELATED APPLICATIONS

This application claims priority to U.S. Provisional Patent Application Ser. No. 62/899,474 entitled IMPLANTABLE REPAIR DEVICES and filed Sep. 12, 2019 and U.S. Provisional Patent Application Ser. No. 62/970,164 entitled IMPLANTABLE REPAIR DEVICES and filed Feb. 4, 2020 which are herein incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This disclosure is related generally to implantable repair devices. More specifically, this disclosure relates to repair devices for repairing bones and joints and especially syndesmotic joints.

### BACKGROUND

It is desirable in orthopedic practice in reducing bone fractures and joint repairs to provide proper compression and stabilization to the joint to support joint regrowth, healing, and mechanical support during the process. Despite decades of surgical progress, many bones and joints do not heal properly. Accordingly, better solutions are needed to improve surgical outcomes of bone and joint surgery. Described herein are solutions needed to improve surgical outcomes of bone and joint surgery, lower the cost of implant parts, make procedures less complicated, provide easier installation and shorter operating room times, resulting in lower costs overall.

### SUMMARY

According to aspects of the disclosure, an implantable device may be provided with first and second bone anchors, at least one suture and a force-generating component. In some embodiments, the first bone anchor is configured to anchor in or on a first bone segment and the second bone anchor is configured to anchor in or on a second bone segment. The at least one suture may interconnect the first bone anchor and the second bone anchor. The force-generating component may be connected to the at least one suture and configured to impart a force thereto. The first bone anchor, the second bone anchor, the at least one suture and the force-generating component may be configured to cooperate together to draw the first bone segment and the second bone segment toward one another with a force in a predetermined range.

In some of the above embodiments, the force-generating component comprises a superelastic material and at least a portion of the predetermined range of force falls within a plateau region of a stress and strain curve of the superelastic material. In some embodiments, the predetermined range of force falls entirely within the plateau region, thereby causing the implantable device to impart a substantially constant force between the first bone segment and the second bone segment even when there is a change in a distance between the bone segments or between the bone anchors. The superelastic material may comprise nitinol.

In some embodiments, the force-generating component is interconnected with the second bone anchor by the at least one suture, and the force-generating component is interconnected with the first bone anchor by a separate second suture. In other embodiments, the force-generating component is interconnected with the second bone anchor by the at least one suture, and the force-generating component is directly connected to the first bone anchor without a suture. The force-generating component and the first bone anchor may be integrally formed. An integrally formed force-generating component and first bone anchor may comprise a superelastic material, and the superelastic material comprises nitinol. The force-generating component may comprise a tube having a helical slit through a wall thickness thereof. In some embodiments, the force-generating component is directly connected to the at least one suture.

In some embodiments, the implantable device further comprises a release tether configured to release the force drawing the first bone segment and the second bone segment toward one another. The implantable device may further include a pair of rings located between the first bone anchor and the second bone anchor. In these embodiments, the release tether may be connected to one of the pair of rings. The pair of rings may be configured to alternately retain the force drawing the first bone segment and the second bone segment toward one another when there is no tension on the release tether, and release the force when there is a tension on the release tether.

According to other aspects of the disclosure, methods of securing bone segments together are provided. In some of the methods, a channel is formed through a first bone segment and at least partially through a second bone segment. An implantable device is provided that may have a first bone anchor, a second bone anchor, at least one suture interconnecting the first bone anchor and the second bone anchor. The implantable device may further include a force-generating component connected to the at least one suture. Some of the methods may further include introducing the second bone anchor through the channel in the first bone segment and at least partially into the channel in the second bone segment. The force-generating component may be introduced at least partially into the channel in the first bone segment. The methods may include locating the first bone anchor in or over a proximal opening in the channel through the first bone segment. In some methods, the at least one suture is drawn in a proximal direction to cause the first bone anchor and the second bone anchor to be drawn toward one another with a force in a predetermined range.

In some of the above methods, the second bone anchor is introduced past two cortical regions of the first bone segment and only one cortical region of the second bone segment. In these methods, the second bone anchor is anchored in a cancellous bone region of the second bone segment. In other methods, the second bone anchor is introduced past two cortical regions of the first bone segment and past two cortical regions of the second bone segment. In these other methods, the second bone anchor is anchored in a distal cortical bone region of the second bone segment.

In some method embodiments, the first bone segment and the second bone segment are interconnected by a syndesmosis. The syndesmosis may be a distal tibiofibular joint. In some embodiments, the first bone segment is a distal fibula and the second bone segment is a distal tibia.

In some method embodiments, the force-generating component comprises a superelastic material, and at least a portion of the predetermined range of force falls within a plateau region of a stress and strain curve of the superelastic material. In other embodiments, the predetermined range of force falls entirely within the plateau region, thereby causing the implantable device to impart a substantially constant force between the first bone segment and the second bone segment even when there is a change in a distance between the bone segments or between the bone anchors. In the above embodiments, the superelastic material may comprise nitinol.

In some method embodiments, the force-generating component is interconnected with the second bone anchor by the at least one suture, and the force-generating component is interconnected with the first bone anchor by a separate second suture. In other embodiments, the force-generating component is interconnected with the second bone anchor by the at least one suture, and the force-generating component is directly connected to the first bone anchor without a suture. The force-generating component and the first bone anchor may be integrally formed. In some embodiments, an integrally formed force-generating component and first bone anchor comprise a superelastic material, which may comprise nitinol.

In some method embodiments, a force-generating component comprises a tube having a helical slit through a wall thickness thereof. Some methods may further include drawing the at least one suture in the proximal direction to cause the helical slit to widen until the first bone anchor and the second bone anchor are drawn toward one another with the force in the predetermined range. In some embodiments, the force-generating component is directly connected to the at least one suture. The method may further include drawing on a release tether of the implantable device to release the force drawing the first bone segment and the second bone segment toward one another. The implantable device may further include a pair of rings located between the first bone anchor and the second bone anchor. The release tether may be connected to one of the pair of rings. The pair of rings may be configured to alternately retain the force drawing the first bone segment and the second bone segment toward one another when there is no tension on the release tether, and configured to release the force when there is a tension on the release tether.

### BRIEF DESCRIPTION OF THE DRAWINGS

A better understanding of the features and advantages of the present disclosure will be obtained by reference to the following detailed description that sets forth illustrative embodiments, in which the principles of the disclosure are utilized, and the accompanying drawings of which:

FIG. 1A is an anterior view of a right foot and ankle showing an exemplary placement of an implantable repair device across a tibiofibular syndesmosis.

FIG. 1B is a lateral view of the foot, ankle and implant of FIG. 1A.

FIG. 2A shows an implantable repair device with a dynamic compression component and a cinch and cinch rings connected to an expandable anchor.

FIG. 2B shows an inserter device that can be used when implanting the repair device of FIG. 2A.

FIG. 3 shows an implantable repair device with a dynamic compression component and a cinch and cinch rings connected to a button anchor.

FIG. 4 shows an implantable repair device with multiple cinch loops connecting a dynamic compression component to two button anchors.

FIG. 5 shows an implantable repair device with multiple cinch loops connecting cinch rings, a dynamic compression component, and two button anchors.

FIG. 6A shows an implantable repair device with a dynamic compression component and a cinch connected to an expandable anchor.

FIG. 6B shows an implantable repair device with a cinch connecting a dynamic compression component, a cinch ring, and an expandable anchor.

FIG. 6C shows an implantable repair device with a cinch connecting a dynamic compression component, a cinch ring, and an expandable anchor.

FIG. 6D shows an implantable repair device with a cinch connecting a dynamic compression component, multiple cinch rings, and an expandable anchor.

FIG. 7A shows a cinch from FIG. 6A with a cinch pin from inside the dynamic compression component with the dynamic compression component removed.

FIG. 7B shows a cinch of FIG. 6B with a cinch pin from inside the dynamic compression component with the dynamic compression component removed.

FIG. 7C shows a cinch of FIG. 6C with a cinch pin from inside the dynamic compression component with the dynamic compression component removed.

FIG. 7D shows a cinch of FIG. 6D with a cinch pin from inside the dynamic compression component with the dynamic compression component removed.

FIG. 8A shows a perspective view of an anchor with four arms and an orifice useful for receiving a cinch.

FIG. 8B shows another view of the anchor shown in FIG. 8A.

FIG. 8C shows a perspective view of an anchor with two arms and an orifice and slot useful for receiving a cinch.

FIG. 8D shows a perspective view of an anchor with four arms and an orifice and slot useful for receiving a cinch.

FIG. 8E shows a perspective view of an anchor assembly with two anchors and two cinch rings.

FIG. 8F shows a side perspective view of the anchor assembly from FIG. 8D with two anchors and two cinch rings.

FIG. 8G shows an exploded view of the anchor assembly from FIG. 8D with two anchors and two cinch rings.

FIG. 9A shows a perspective view of another implantable repair device with a dynamic compression component and a release tether.

FIG. 9B shows a perspective view of the interior components of the device shown in FIG. 9A.

FIG. 9C shows a side view of the interior components of the device shown in FIG. 9A.

FIG. 10 shows a perspective view of another implantable repair device with a dynamic compression component and a release tether.

FIG. 11A shows a perspective view of another implantable repair device with a dynamic compression component located between two button type anchors.

FIG. 11B shows an exploded view of the components of the device shown in FIG. 11A.

FIG. 12A shows a side view of an inserter for inserting an implantable repair device.

FIG. 12B shows a close up view of the tip of the inserter shown in FIG. 12A.

FIG. 12C shows a close up view of the tip of the inserter shown in FIG. 12B rotated 90°.

FIG. 13 shows a typical stress-strain diagram for a shape memory alloy/superelastic material.

FIG. 14 shows a typical stress-strain diagram for a shape memory alloy/superelastic material compared with other materials.

### DETAILED DESCRIPTION

Described herein are implantable repair apparatuses (devices and methods), particularly for repairing bones and joints such as syndesmotic joints. Also described herein are inserters useful for implanting the repair devices. The devices may be especially useful for preventing the bone or joint from weakening or moving over time. For example, described herein are devices and methods that include a dynamic compression component, one or more cinches, and one or more bone anchors and/or anchor buttons. Some devices and methods may also include one or more cinch rings. The devices may be configured for inserting through an incision or break in the skin and into or through a bone or connective tissue. The devices and methods may be especially useful for repairing or stabilizing a syndesmotic joint. For example, the devices and methods may be useful for repairing or stabilizing a movable fibrous joint connecting the tibial and fibular bones in the lower leg. The devices may be especially useful for maintaining a desired tension during a healing process and achieving a better or more functional bone or joint alignment than currently available.

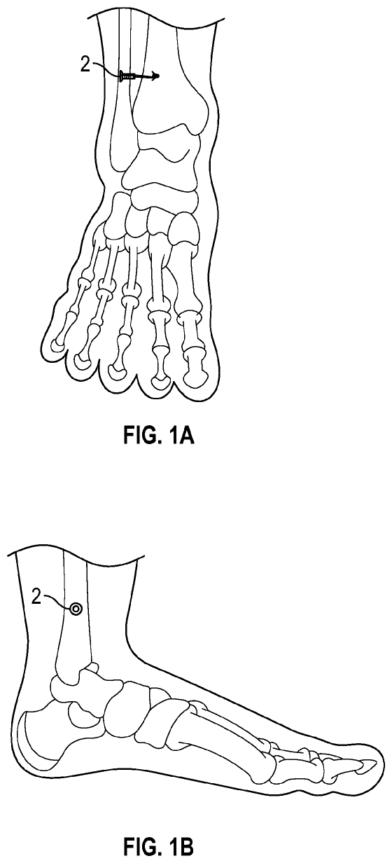

FIG. 1A is an anterior view of a right foot and ankle showing an exemplary placement of an implantable repair device across a tibiofibular syndesmosis according to aspects of the present disclosure. FIG. 1B is a lateral view of the foot, ankle and implant of FIG. 1A. In this exemplary embodiment, repair device 2 is inserted in a lateral to medial direction across the fibula, across the intervening syndesmosis and partially through the tibia to provide a substantially constant force between the fibula and the tibia, such as for healing of the syndesmosis. With this placement, device 2 crosses a lateral cortical region of the fibula, a middle cancellous region, a medial cortical region of the fibula, a portion of the syndesmosis, a lateral cortical region of the tibia, and anchors in a cancellous region of the tibia. In short, the repair device 2 crosses three cortical regions in this implementation, with a proximal anchor positioned on cortical bone and a distal anchor positioned in cancellous bone. In other implementations, the implant may extend through the tibia (i.e. through a total of four cortical bone regions) and have both proximal and distal anchors positioned on cortical bone. In some implementations, the implant may extend only partway through the fibula and tibia once it is implanted (i.e. across a total of two cortical bone regions) and have both proximal and distal anchors positioned in cancellous bone. In some implementations, a medial to lateral approach may be taken, starting in the tibia and going part way or all the way through the fibula. In some implementations, the lateral anchor can be introduced from the lateral side and the medial anchor can be introduced from the medial side and the two portions of the implant connected together in situ, rather than both anchors being introduced from one side. In some implementations, there may be more, less or no syndesmosis between the bone segments where the implant is placed. In some implementations, more than one implant is placed, such as two implants across the tibiofibular syndesmosis, one directly above the other.

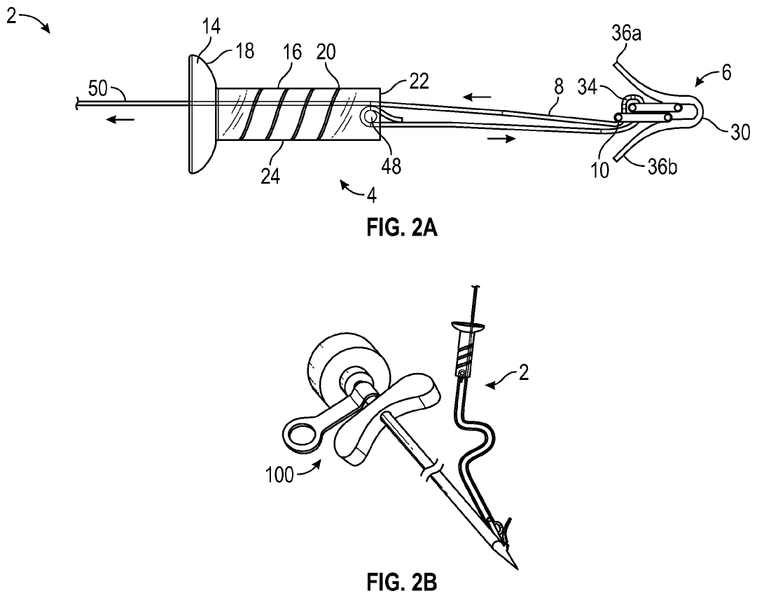

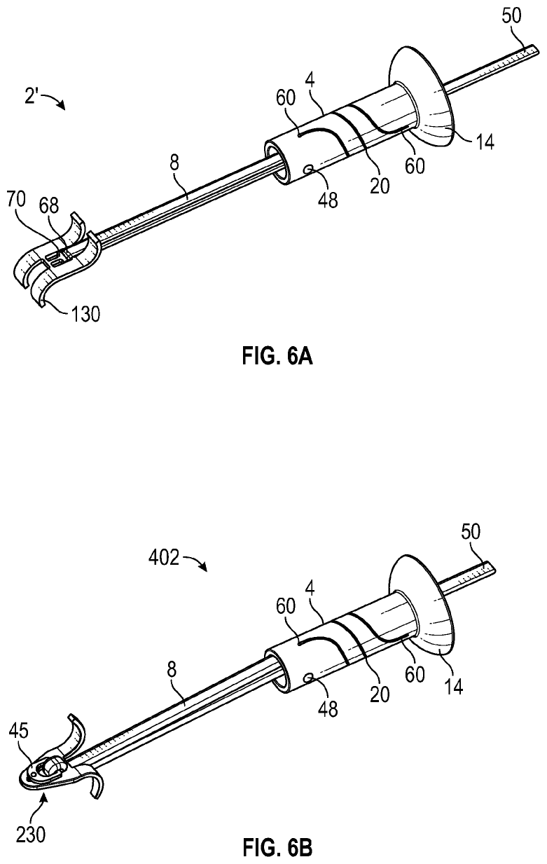

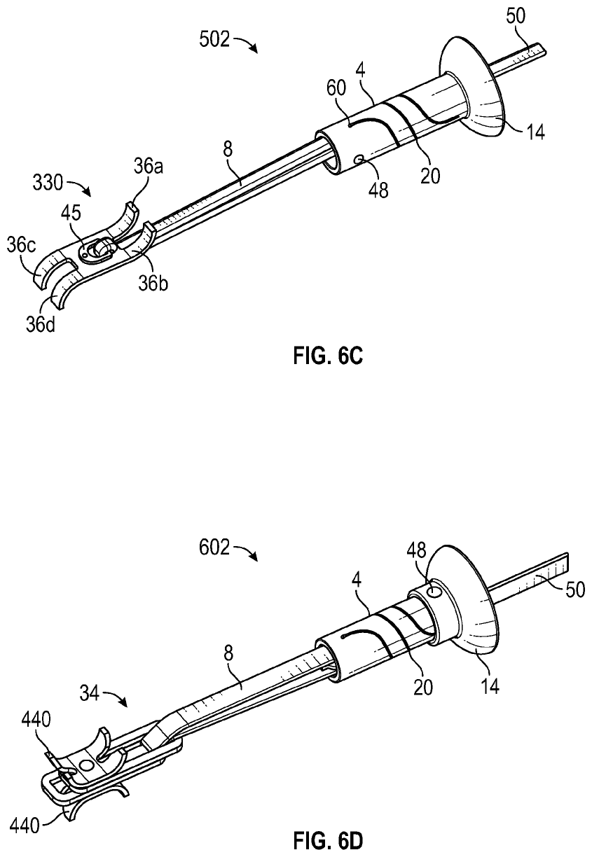

FIG. 2A shows an exemplary embodiment of an implantable compression device 2 (also referred to as an implantable repair device) with a dynamic compression component 4 (also referred to as a force-generating component), bone anchor assembly 6, suture loop 8, and cinch assembly 34. The dynamic compression component 4 includes head region 14 and body 16. Body 16 is tube-shaped with an outer wall 24 around a hollow center 22. A feature 20 is formed in the outer wall 24 of the body 16 and may be configured to allow dynamic movement such as stretching and contracting of the dynamic compression component 4. The feature 20 may be a cut-out that extends inwards to the hollow center 22 (e.g., the feature 20 may extend all the way through the outer wall 24). The dynamic compression component 4 may comprise or be formed of nitinol (NiTi), another shape memory alloy, superelastic, elastomeric and/or shape-changing material that allows the body 16 to move (e.g., stretch and contract). In some variations, the body 16 is configured to contract and extend longitudinally. In some variations, the body 16 is in addition or instead configured to contract and expand radially. In some variations, the feature 20 may include two or more helices, partial helices, lines, or other shapes that allows the dynamic compression component 4 to contract and extend longitudinally or contract and expand radially. For example, the dynamic compression component 4 may include a generally helically shaped feature with a tail extension 60 (see FIGS. 6A-6D) and the dynamic compression component 4 may be configured to both contract and extend longitudinally and to contract and expand radially. The body 16 and the dynamic compression component 4 may not uniformly change (e.g., contract and extend longitudinally or radially contract and expand). Different portions of the body 16 may change to different extents. For example, the region near the helix may change shape more than does an end region.

The head 14 of the dynamic compression component 4 may be configured to fit over or against a first end of body 16. The head 14 may be configured to oppose part of a bone or other hard object and hold the dynamic compression component 4 and body 16 in place against the bone. The head 14 may be made of ceramic, nitinol, polyether ether ketone (PEEK), polyaryl ether ketone, ultra-high molecular weight polyethylene (UHMWPE), or another biocompatible material. The head 14 may be made from the same material as the body 16 or may be made from a different material than the body 16. A bone plate (not shown) may be present (between the head 14 and a bone) and the head 14 may be made from the same material as the bone plate or may be made from a different material than the bone plate. The head 14 may be made from a non-conductive material that minimizes or prevents corrosion that may otherwise occur when two metal pieces contact one another such as if a metal head contacted a metal body or metal bone plate. In some variations, the head 14 is made from PEEK or UHMWPE and the body 16 is made from nitinol. The head 14 may separate the body 16 from a bone plate and prevent corrosion. The head 14 may be held in place relative to and/or against body 16 with a retainer 18. Retainer 18 may be a pin. Retainer 18 may include one, two, or more parts, such as one pin or more than one pins. An adhesive, cement, or screw may also or instead be used to hold the head 14 in place relative to the body 16. The head 14 in FIG. 2A is hemispherical (has rotational symmetry) to allow the implantable compression device 2 to be implanted in any orientation. Device 2 may be used in conjunction with an implantable plate having a through-hole or holes with a hemispherical recess to mate with head 14, thereby allowing dynamic compression component 4 to enter the bone at a non-perpendicular angle. In some variations, the head may have an alternate geometry (e.g., pentagonal, octagonal). In some embodiments, head 14 may sit directly on an outer surface of the bone, or may sit within a countersink in the bone such that the proximal surface of head 14 sits generally flush with the outer surface of the bone when implanted. The countersink may formed by a separate countersink tool or may be formed by head 14 itself. In other embodiments, a counterbore may be formed in the bone so that head 14 may be recessed into the bone.

FIG. 2A shows that in this exemplary embodiment, the implantable compression device 2 also includes a bone anchor assembly 6. The bone anchor assembly 6 includes anchor 30 and cinch assembly 34. The anchor 30 includes a bone anchor arm 36a, anchor body 38, and anchor head 40. The anchor 30 is formed of nitinol (NiTi) or another elastomeric or shape-changing material that allows the anchor 30 to change shape and move the bone anchor arm 36a from a first position to a second position so that the bone anchor arm 36a locks the implantable compression device 2 in place such as in place inside cancellous bone. FIG. 2A shows a second bone anchor arm 36b. The anchor 30 changes shape and moves the second bone anchor arm 36b from a first position to a second position so that the second bone anchor arm 36b helps lock the implantable compression device 2 in place such as in place inside cancellous bone. The two or more bone anchor arms may move in the same direction or in opposite directions.

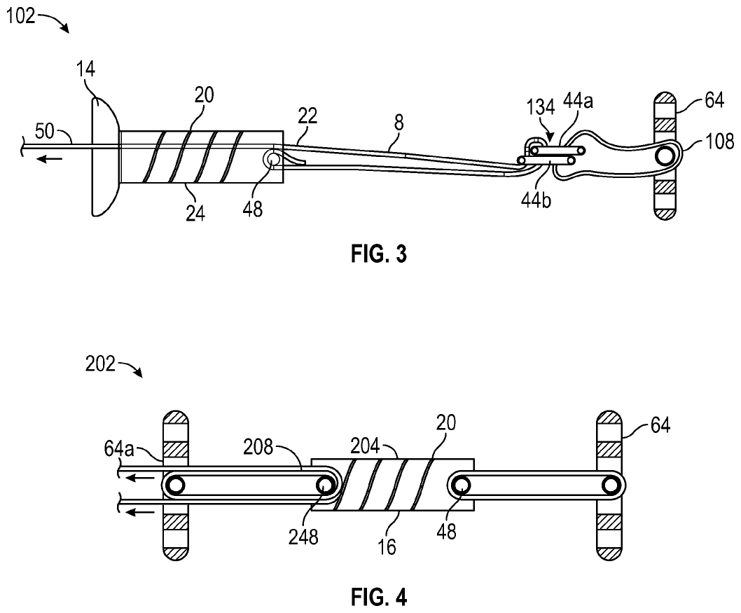

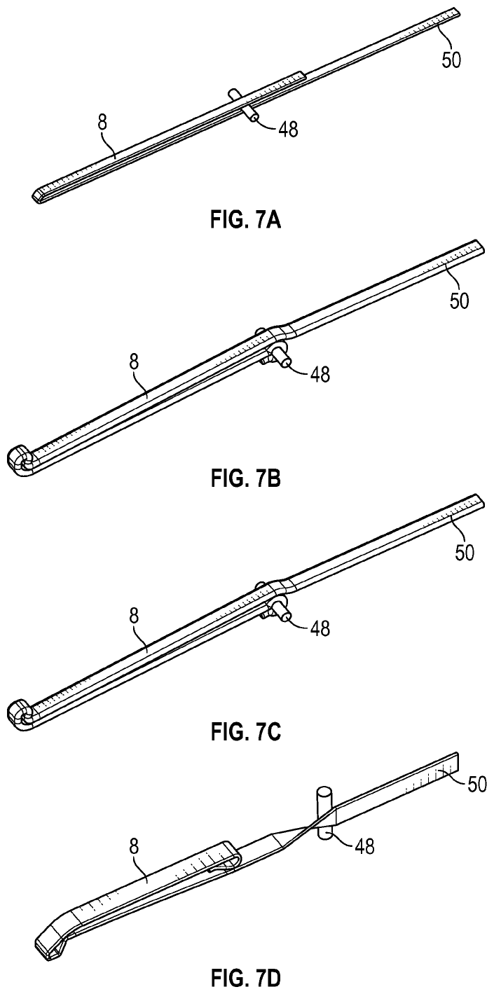

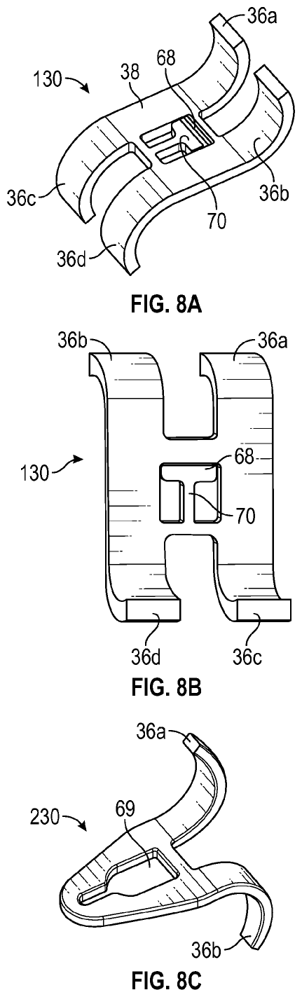

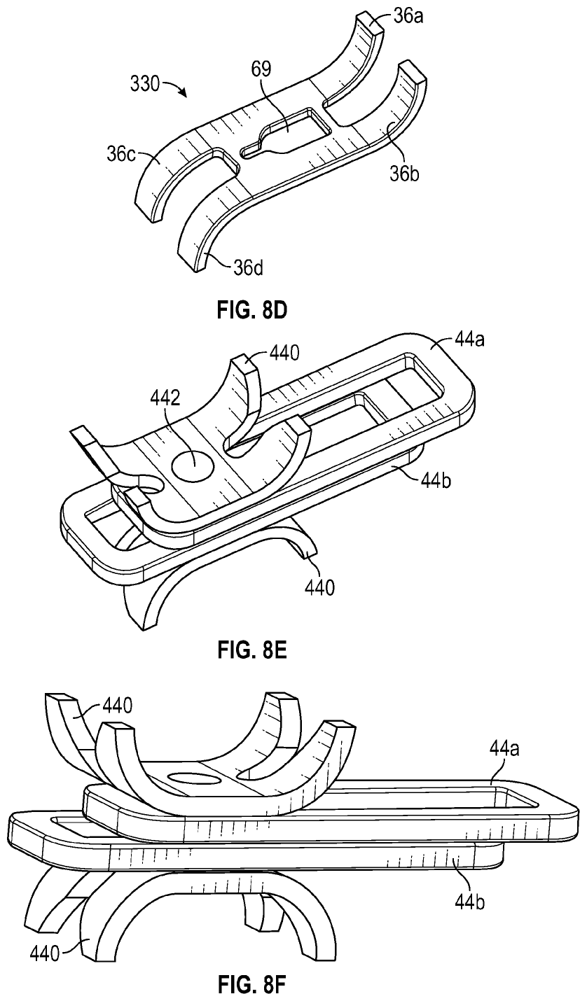

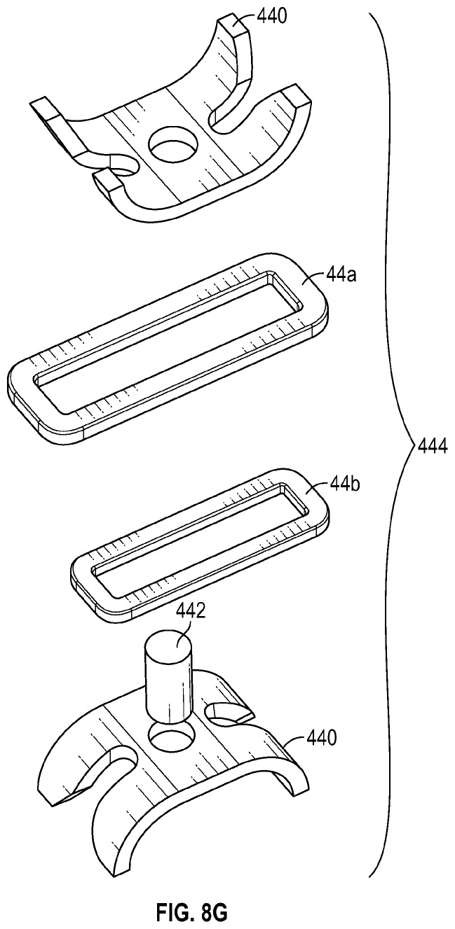

Cinch assembly 34 includes a first cinch ring 44a and a second cinch ring 44b (as best seen in FIG. 3.) The first cinch ring 44a and the second cinch ring 44b have an opening to allow a strand of suture loop 8 to pass through the opening and around an end of the cinch ring. The first cinch ring 44a and the second cinch ring 44b together tension suture loop 8 when a force is placed on the first suture end 50 of suture loop 8. (See also FIG. 6D, FIG. 7D, and FIGS. 8D-8F). Cinch loop 8 extends from the first suture end 50, through the head 14, through the hollow center 22 of body 16, and then wraps around the first cinch ring 44a and the second cinch ring 44b and is anchored to cinch prong 48 on body 4. Prong 48 may be press fit, laser welded or connected by other means to body 4.

According to one exemplary implementation when in use to repair a syndesmotic joint or perform another repair in a body of a patient, the dynamic compression component 4 is expanded into an extended condition when installed. A hole is first made through bone such as with a bone drill. For example, a hole may be made through the distal end of a fibula and at least partially through the adjoining tibia when repairing a distal tibiofibular syndesmosis. Anchor assembly 6 is then placed through the fibula and inside the hole in the tibia with the first suture end 50 and head 14 remaining outside the fibula. As shown in FIG. 2B, an inserter device 100 may be used to hold the anchor of device 2 in a retracted state while pushing the anchor into the holes in the fibula and tibia. The implantable compression device 2 may be used to cross a broken bone or other damaged connective tissue in need of repair. As indicated above, the anchor 30 may be a shape-changeable bone anchor. The anchor 30 is activated to move the first bone anchor arm 36a in place against the inside of the bone and anchor the implantable repair device 2 inside the bone. The anchor 30 activation also moves the second bone anchor arm 36b in place against the inside of the bone and anchors the implantable repair device 2 inside the bone. In some variations (see e.g., FIGS. 6A, 6C, and 6D) the anchor activation also moves additional bone anchor arms in place against the inside of the bone to anchor the implantable repair device 2 inside the bone. The anchor activation may be accomplished by a temperature change, releasing the anchor arms from an inserter device 100 and/or by other means. The anchor arms may have different configurations and may be on the same end of the anchor 30 or may be on different ends or may be on other parts of the anchor 30. In some variations, an implantable repair device 2 may have multiple anchors with multiple arms.

Generally after the anchor 30 is anchored in the bone, the first suture end 50 is pulled, tensioning the suture loop 8. The suture loop is fastened and the implantable compression device is in place in the bone. In the exemplary implementation mentioned above, once repair device 2 is implanted and tensioned, it exerts a dynamic force that urges the distal ends of a fibula and tibia together. The wound is then closed. Over time, the suture material may stretch. However, the dynamic compression component 4 such as made from shape-memory or other material continues to contract over time as the suture material stretches or as the surrounding bone compresses, maintaining a desired tension on the implant. In some embodiments, repair device 2 is configured and tensioned such that it remains in a plateau region of the shape-memory strain-stress curve during use. As such, repair device 2 is able to provide a generally constant tension on the surrounding bone as the bone moves, the bone contracts, the sutures stretch, the device heads migrate into the bone and/or other changes occur over time. As the dynamic compression component 4 (e.g., the shape-memory or other material) can contract until the feature 20 contracts to its smallest closed form (e.g., a beginning form) the configuration of the feature 20 may be chosen to provide a desired amount of contraction over time. For example, the feature 20 may include one or more than one helix or other shaped feature. Two helices or other shaped features may be in the same orientation (e.g., parallel) or in different orientations, such as opposite orientations. A helix or other shaped feature may extend partway through the wall but in general extends all the way through the wall. A helix or other shaped feature may contain from 1-15 turns, such 1 turn, 2 turns, 3 turns, or more than 3 turns. The feature 20 (in the dynamic compression component 4) may contain 1 helix or other shaped feature, 2 helices or other shaped feature, 3 helices or other shaped feature, or more than 3 helices or other shaped feature, (e.g., these helices or other shaped features may be separate). Multiple features 20 may be located in the same region of body 4 such that they are interlaced or interdigitated, and/or the features may be separated longitudinally on body 4. A helix may have a pitch (turns per mm) of 0.1 turns per mm to 10 turns per mm or anything in between these amounts, such as between 0.5 turns per mm and 3 turns per mm. In some variations the pitch is about 1 turn per mm A helix or other shaped feature may have a kerf value (cut width) between 0.01 mm to 1 mm such as between 0.05 mm and 0.5 mm or 0.08 mm and 0.3 mm. The feature 20 may be configured or chosen to substantially return from a second shape to a first shape in a particular time frame or to a particular degree. In some variations, a feature may be configured to substantially return from a second shape to a first shape in 1 day, 3 days, 7 days, 14 days, 28 days or more than 28 days. In some variations, a feature may be configured to substantially return from a second shape to part way (e.g., 50%) to a first shape in 1 day, 3 days, 7 days, 14 days, 28 days or more than 28 days.

FIG. 3 shows another implantable compression device 102. This device is similar to the implantable compression device 2 shown in FIG. 2A but has a button 64 rather than a bone anchor at the distal end of the implantable compression device 102. The button 64 is configured to rest against (on the outside of) a second side of a bone (such as the far side of a tibia) and to hold the implantable compression device 102 in place. In some embodiments, a hole is drilled through both a fibula and a tibia. In use, button 64 is aligned with the holes so that it may be passed through both bones. Once it emerges from the far side of the tibia, it may be rotated 90 degrees (such as with suture(s), instrumentation or just pulling back on cinch loop 8) so that it rests on the far surface of the tibia. In some implementations, the insertion and rotation of button 64 may be accomplished entirely from the proximal/fibula side of the syndesmosis without making any incision on the distal/tibia side of the joint. Additionally, a cinch assembly 134 remains inside the bone (in some embodiments inside the tibia, fibula or between the bones) and is separated from the button 64. The cinch assembly 134 has a first cinch ring 44a and second cinch ring 44b. The implantable compression device 102 includes a cinch loop 8. Cinch loop 8 extends from the first suture end 50, through the head 14, through the hollow center 22 of body 16, and then wraps around the first cinch ring 44a and the second cinch ring 44b and is anchored to cinch prong 48. The implantable compression device 102 also includes a second cinch loop 108. The second cinch loop 108 connects the distal ends of first cinch ring 44a and the second cinch ring 44b with the openings in button 64 and is configured to tension the button 64 to the cinch assembly 134. Similar to as described for the implantable compression device 2 shown in FIG. 2A, pulling on the first suture end 50 tightens the implantable compression device 102.

FIG. 4 shows another implantable compression device 202. This device is similar to the implantable compression device 102 shown in FIG. 3 but has a second button 64a configured for placement on a proximal side of the bone so that the implantable compression device 202 includes buttons on both ends. When in place in a body, the bone segments have a first button 64 on the distal side and a second button 64a on the proximal side of the bone(s). The dynamic compression component 204 has a body 16 with feature 20 configured for expanding and contracting to provide dynamic compression to the bone(s). The dynamic compression component 204 includes first cinch prong 48 as well as a second cinch prong 248. The cinch 208 connecting the second cinch prong 248 and the second button 64a has two ends that can be pulled. When the implantable compression device 202 is inside a bone and the ends pulled, cinch 208 is tightened. In some implementations, cinch 208 is tied off against button 64a after being tightened. The implantable compression device 202 is configured to be pulled through a hole in a bone in either direction.

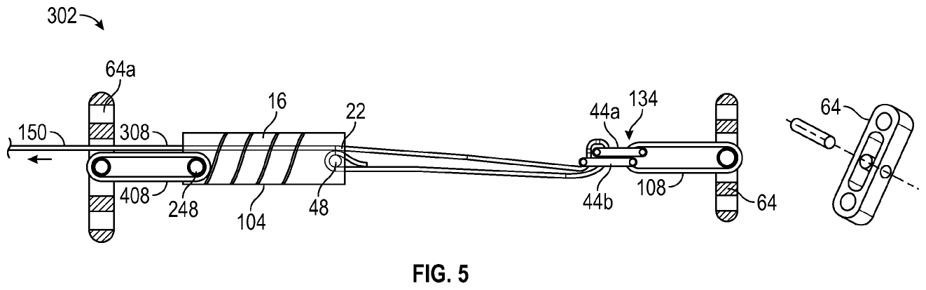

FIG. 5 shows another implantable compression device 302. This device is similar to the implantable compression device 202 shown in FIG. 4 but is configured with cinch assembly 134 and three cinches. In this device, a cinch 308 (middle cinch) that extends proximally is configured to tension the device. Cinch 308 extends from the first suture end 150, through the second button 64a, through the hollow center 22 of body 16, and then wraps around the first cinch ring 44a and the second cinch ring 44b and is anchored to cinch prong 48. Pulling on the first suture end 150 tensions the cinch 308 and the implantable compression device 302. A cinch 108 (distal cinch) loops and holds the first button 64 with the first cinch ring 44a and the second cinch ring 44b of the cinch assembly 134. A cinch 408 (proximal cinch) loops and holds the second button 64a to the dynamic compression component 104 by the cinch prong 248. As can be seen by the inset diagram in FIG. 5, buttons 64 and 64a may have an elongated aspect ratio. This allows the buttons to pass through a smaller diameter hole through the bone segments but have a larger footprint once rotated into place on the outside of the bone. In some embodiments, the buttons 64 and 64a have a length that is at least 2 times their width, at least 3 times their width, at least 4 times their width, or more than 4 times their width.

FIG. 6A shows a perspective view of an implantable compression device 2′ such as the device shown in FIG. 2A. FIG. 8A and FIG. 8B show different views of the anchor 130 shown in FIG. 6A. The anchor 130 includes an orifice 68 configured to accept a cinch loop therethrough. The anchor 130 also includes a T-bar 70. The orifice 68 and the T-bar 70 cooperate to allow one-way movement of the suture of the cinch loop therethrough. FIG. 7A shows the cinch loop 8 and cinch prong 48 of the implantable compression device 2′. The anchor 130 (see also FIG. 8A and FIG. 8B) also includes four anchor arms 36a, 36b, 36c, and 36d. In some variations, the anchor arms may all be substantially the same while in other variations one or more arms may be different from the others. Each arm may be configured (pre-set) to bend. The arms may be configured (pre-set) to bend in the same or opposite directions. In some implementations, anchor 130 rotates when cinch 50 it tightened to engage anchor arms 36a, 36b, 36c, and 36d into the surrounding bone.

FIG. 6B shows a perspective view of an implantable compression device 402. The anchor 230 (see also FIG. 8C) has a first anchor arm 36a and a second anchor arm 36b. The anchor 230 also has an orifice 69 (shown in FIG. 8C) configured to accept an end of the cinch 8. The implantable compression device 402 also includes a cinch 8 that extends through orifice 69 and wraps around an end of a slidable cinch ring 45. Cinch ring 45 may be provided with a pin as shown in FIG. 6B with an enlarged head (not shown) on the bottom side of anchor 230 to slidably retain cinch ring 45 on the anchor. The pin is retained in a narrow portion of orifice 69 (shown in FIG. 8C.) FIG. 7B shows the cinch loop 8 and cinch prong 48 of the implantable compression device 402 shown in FIG. 6B. The cinch 8 is wrapped around cinch prong 48 of the implantable compression device 402. Proximal end 50 of cinch 8 is configured to be pulled to tighten cinch 8.

FIG. 6C shows a perspective view of an implantable compression device 502. The implantable compression device 502 is similar to the implantable compression device 402 shown in FIG. 6B except that the anchor 330 (also shown in FIG. 8D) has four anchor arms and a single orifice 69 (shown in FIG. 8D) through the anchor body. A slidable cinch 45 is attached to the anchor with a pin as previously described in reference to anchor 230 shown in FIGS. 6B and 8C. FIG. 7C shows the cinch loop 8 and cinch prong 48 of the implantable compression device 502 shown in FIG. 6C.

FIG. 6D shows a perspective view of an implantable compression device 602. The implantable compression device 602 has a cinch assembly 34 and two anchors 440 that slide together relative to cinch assembly 34. FIG. 7D shows the cinch loop 8 and cinch prong 48 of the implantable compression device 602 shown in FIG. 6D. Another cinch ring (not shown in FIG. 6D or FIG. 7D) resides inside body 4 and connects the distal end of cinch 8 with the vertically oriented cinch prong 48. FIG. 8E and FIG. 8F show perspective views of the cinch assembly and two anchors 440. FIG. 8G shows an exploded view of the cinch assembly and two anchors 440. In this embodiment, the two anchors 440 are rigidly attached to one another by pin 442. Anchors 440 may be press-fit onto pin 442, laser welded thereto, or attached to one another by other suitable means. Once assembled, cinch rings 44a and 44b are slidably captivated between the two anchors 440 by pin 442, and the five components together make up anchor assembly 444.

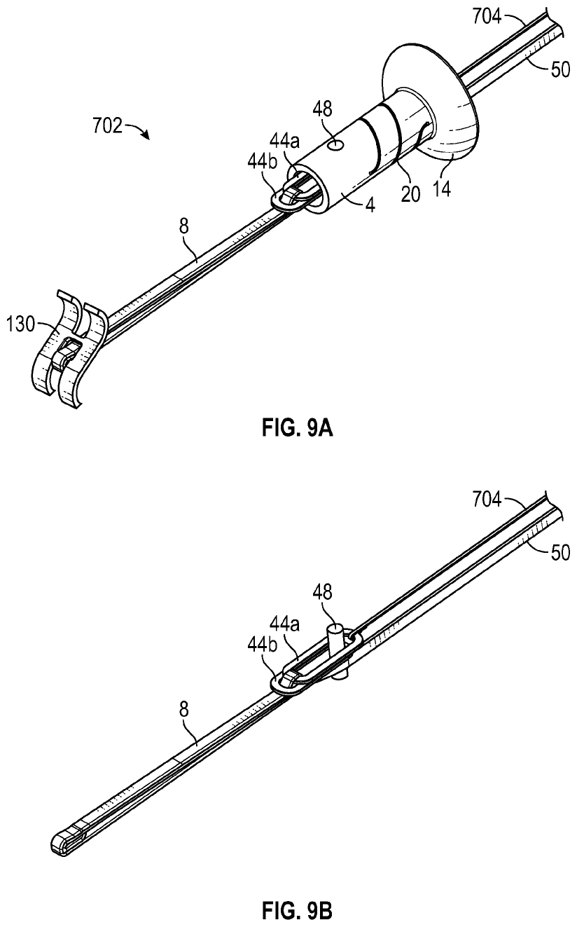

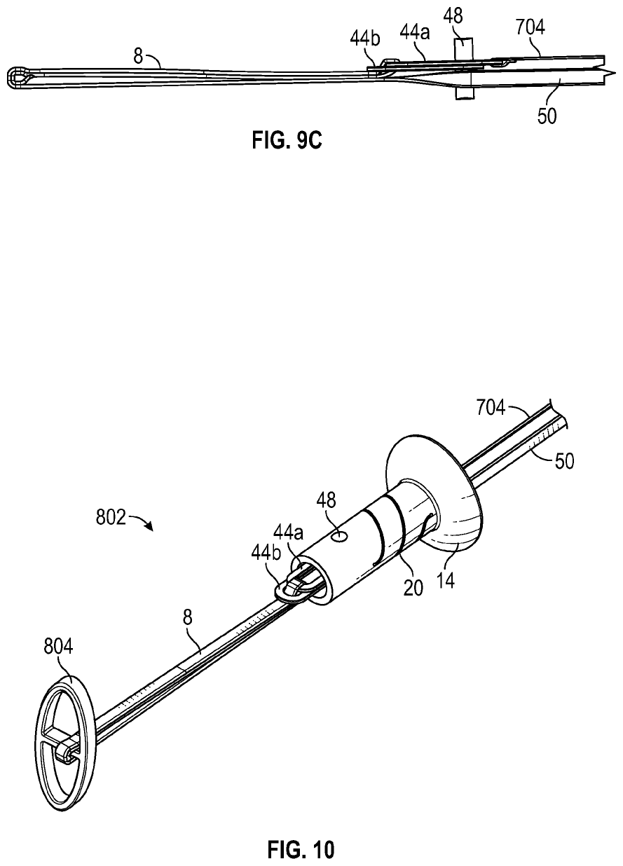

FIG. 9A shows a perspective view of an implantable compression device 702. The implantable compression device 702 is similar to device 2′ shown in FIG. 6A, however device 702 includes a release tether 704 configured to release the tension on cinch loop 8 after it has been tightened so that compression device 702 can be removed for repositioning or explanted after the bone joint has healed. FIG. 9B is a perspective view of compression device 702 with body 4 and anchor 130 removed so that the other components of device 702 can be more readily seen. FIG. 9C is an enlarged partial side view of the components shown in FIG. 9B. As shown in FIGS. 9B and 9C, release tether 704 may be attached to the proximal end of cinch ring 44a. When tether 704 is pulled proximally (i.e. to the right in these figures), a larger gap between the distal ends of cinch rings 44a and 44b is created, allowing cinch loop 8 to lengthen and release tension. The compression device may then be removed by continuing to pull proximally on release tether 704, and or cinch loop 8 may then be re-tensioned by releasing tether 704 and pulling proximally on cinch portion 50.

FIG. 10 shows a perspective view of an implantable compression device 802. The implantable compression device 802 is similar to device 702 shown in FIG. 9A, however device 802 utilizes a button type anchor 804 instead of the prong type anchor 130 of device 702. The button type anchor 804 is configured to be rotated into place against the outside surface of cortical bone tissue after the anchor 804 emerges from a hole through the bone. In contrast, the prong type anchor 130 shown in FIG. 9A is better suited for engaging with cancellous bone tissue located inside the bone. As with the buttons show in FIG. 5, button 804 may be provided with a length that is greater than its width to allow it to pass through a smaller diameter hole through the bone segments but have a larger footprint once rotated into place on the outside of the bone. In some embodiments, button 804 has a length that is at least 2 times its width, at least 3 times its width, at least 4 times its width, or more than 4 times its width. Compression device 802 may also be provided with a release tether 704 as shown.

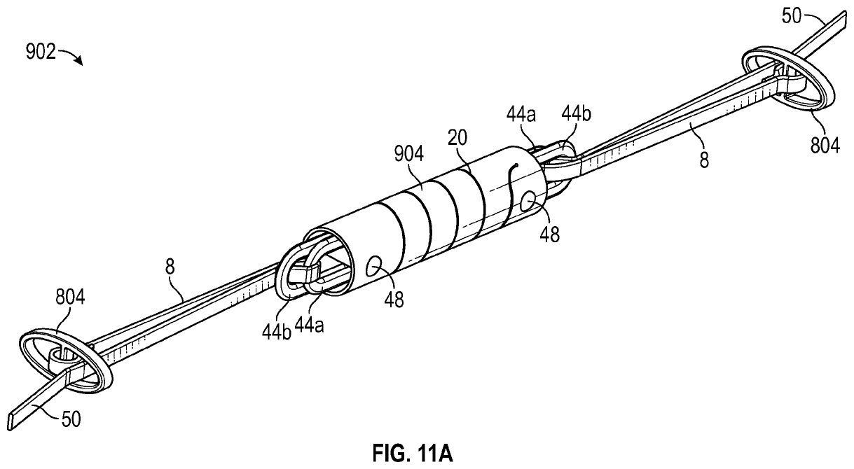

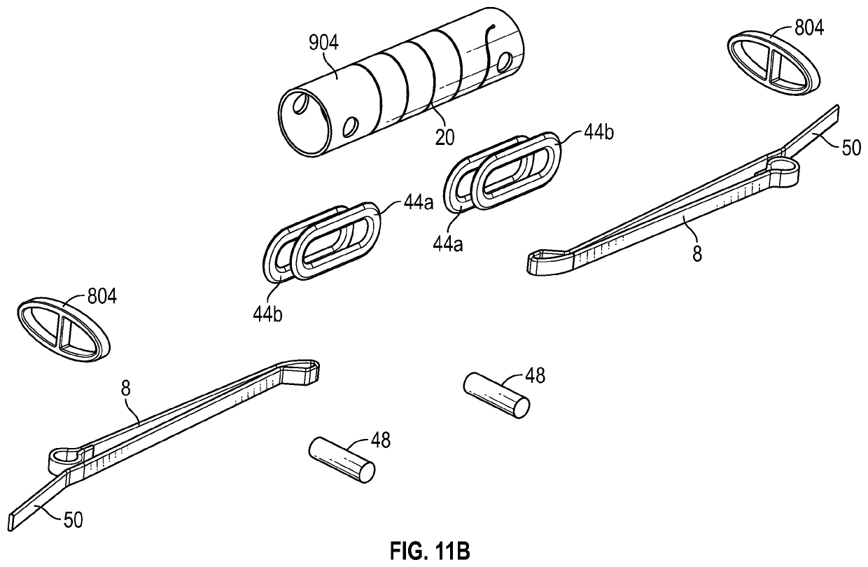

FIG. 11A shows a perspective view of an implantable compression device 902. The implantable compression device 902 is similar to device 802 shown in FIG. 10, however device 902 utilizes a button type anchor 804 at both ends of the device. The dynamic compression/force-generating component 904 of device 902 is similar to the dynamic compression component 204 of device 202 shown in FIG. 4. Device 902 includes a pair of cinch rings 44a and 44b located at each end of force-generating component 904, thereby allowing tension to be applied at either or both ends of device 902. FIG. 11B is an exploded view of device 902 showing further details of each of its components.

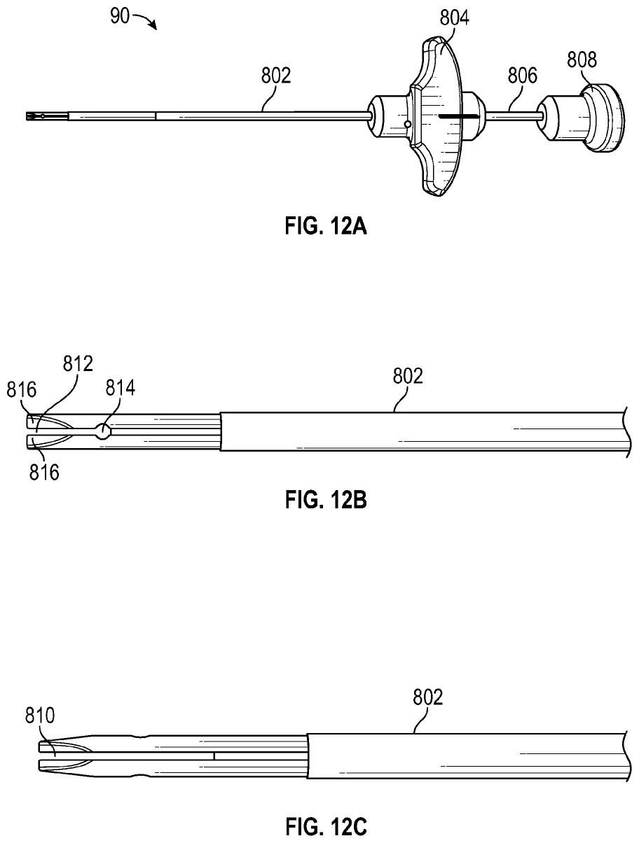

FIG. 12A shows a top view of an inserter 90 for inserting an implantable repair device. FIG. 12B shows a close up of the tip of the inserter 90 shown in FIG. 12A. FIG. 12C shows a close up view of the tip of the inserter 90 shown in FIG. 12B rotated 90°. As seen in FIG. 12A, inserter 90 comprises an outer tube 802 coupled to a handle 804, and a pushrod 806 coupled to a knob 808. Pushrod 806 is slidably received within outer tube 802. Slot 810 in the distal end of outer tube 802 (shown in FIG. 12C) is configured to slidably receive an anchor, such as anchor 38 shown in FIG. 8A. In some embodiments, outer tube 802 serves to hold anchor arms 36a, 36b, 36c and 36d in a flat, retracted configuration while being inserted in bone. In some embodiments, the anchor is cryogenically cooled before it is loaded into inserter 90. Slot 812 and orifice 814 may be provided in the distal end of outer tube 802 (as shown in FIG. 12B) to allow a suture to pass through the anchor and the outer tube 802 when the anchor is being inserted. The distal end of outer tube 802 may include bevels 816 in at least one dimension to aid in navigating the inserter and anchor through bone.

In use, inserter 90 may be used to push an anchor and an attached suture through a hole in a bone or bones. When the anchor is at the desired depth in the bone (as may be confirmed with imaging) and is ready to be deployed, knob 808 and pushrod 806 may be held in a stationary position while handle 804 is retracted proximally, thereby pulling outer tube 802 back relative to pushrod 806 and ejecting the anchor from slot 810 in the distal end of outer tube 802. In some embodiments, a locking element (not shown) may be provided between handle 804 and pushrod 806 to keep the anchor from being deployed until it is fully inserted in the bone and the locking element is removed.

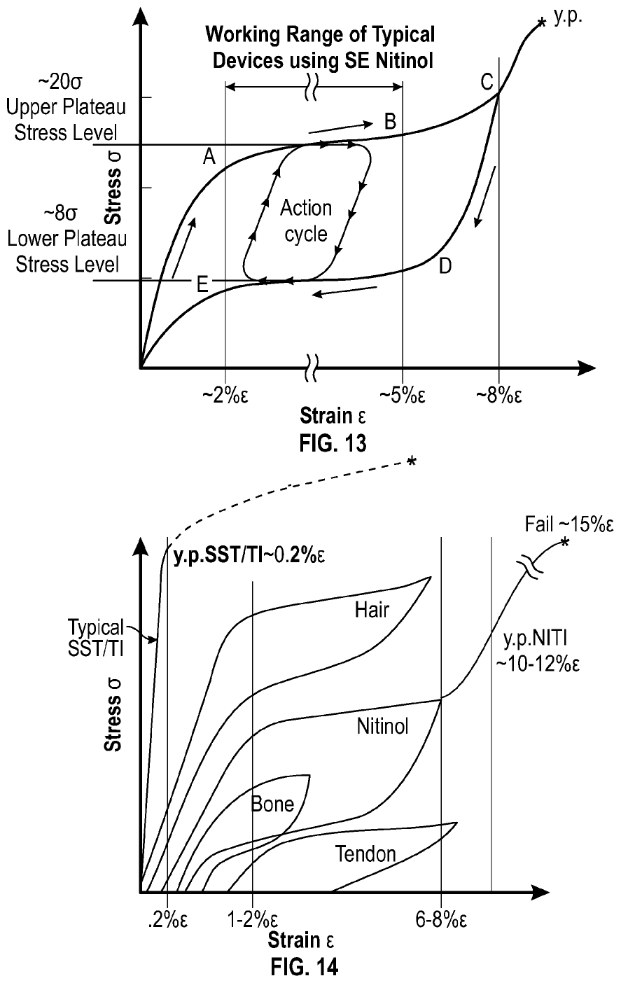

FIG. 13 shows a typical stress-strain diagram for a shape memory alloy/superelastic material. The upward arrows indicate regions where there is a forward martensite transformation in the material as strain on it is being increased, and the downward arrows indicate regions where there is an inverse martensite transformation as the strain is being unloaded. The superelastic material exhibits two plateau regions: an upper plateau region between points A and B, and a lower plateau region between points D and E. In both of these plateau regions the material exhibits a fairly stable stress as the strain varies over a wide range.

When an implantable device as disclosed herein includes a force-generating component formed from a superelastic material and it is implanted and tensioned as previously described, its performance will generally fall along the curve of FIG. 13. The force exerted by the force-generating component will typically start at initial value but will change as the bone segments move closer together or farther apart, the bone segments compress, grow, remodel or otherwise heal, the anchors migrate in the bone, the sutures or other components stretch or change in dimension, etc. According to aspects of the present disclosure, an implantable device may be configured and installed such that its initial tension and the expected tension over its operable life falls within a predetermined range, and at least a portion of that predetermined range falls within one of the two plateau regions of the stress and strain curve. This advantageously allows the force-generating component to continue to deliver a fairly constant force as the bone segments heal. In some embodiments, the predetermined range of force falls entirely within the plateau region, thereby causing the implantable device to impart a substantially constant force between the first bone segment and the second bone segment even when there is a change in a distance between the bone segments or between the bone anchors.

In some embodiments, a cinch loop of an implanted device is tensioned to a target force or range of force located on the upper plateau between points A and B. It may be desirable to select a force closer to or at point B since the bone segments and or implant are more likely to contract than to expand. The implant may be designed such that the force that it imparts to the bone segments does not drop below point A on the stress-strain curve.

In some embodiments, a cinch loop of an implanted device is tensioned to a target force or range of force located on the lower plateau between points D and E. This may be accomplished by first tensioning the implant with a higher force, such as the force associated with point C on the stress-strain curve, and then releasing some of the force to arrive at the lower plateau region. It may be desirable to select a force closer to or at point D since the bone segments and or implant are more likely to contract than to expand. The implant may be designed such that the force that it imparts to the bone segments does not drop below point E on the stress-strain curve.

In some embodiments, an implant constructed and implanted according to aspects of the present disclosure goes through the action cycle shown in FIG. 13 as the bone segments and implant components move relative to one another.

In some embodiments, the implant is configured to deliver a substantially constant force to the bone segments that does not vary more than 2% over the operable life of the implant. In other embodiments, the implant is configured to deliver a substantially constant force to the bone segments that does not vary more than 5%, 10%, 20%, 30%, 50% or 70% over the operable life of the implant. In some embodiments, a #2 H.S. ultra high molecular weight polyethylene (UHMWPE) suture material is used having a failure strength of about 50 lbs. of tension. A useful working range of 10 to 25 lbs. may be used with this suture in some embodiments. In these embodiments, depending on the number of suture loops between implant components, forces of 10 to 25 lbs. (one strand), 20 to 50 lbs. (two strands), 30 to 75 lbs. (three strands), 40 to 100 lbs. (four strands) or more may be applied to the bone segments. In some embodiments, less than 10 lbs. of force may be applied to the bone segments. A calibrated tensioning device may be used by surgeons to ensure a predetermined tension or range of tension in the implant suture is obtained.

FIG. 14 shows a typical stress-strain diagram for a shape memory alloy/superelastic material compared with typical curves for titanium, stainless steel and three biological materials (hair, bone and tendon.) As can be seen, Nitinol exhibits a stress-strain curve that is similar to those of the biological materials. In particular, the Nitinol curve overlaps with the bone curve. In some embodiments, by designing a helix cut, force-generating component wall thickness and diameter appropriately, the implant can match the properties of the bone into which it is being implanted.

In other embodiments, a force-generating component may be formed from a resilient material that is not superelastic, thereby performing like a simple spring. In these embodiments, the force-generating component provides a force that is generally proportional to the amount it is being stretched at the time. For example, a force-generating component may be formed from a stainless steel, titanium, polyether ether ketone (PEEK), other material or combinations thereof.

As indicated above, a cinch may be a loop or may have one or more free ends. In some variations, one end may be fasted or looped around a retainer or pin to hold the cinch in place. In some variations, more than one end of a cinch is free and available for pulling by a medical practitioner in order to tension the cinch and repair device. A cinch may be made from a suture material, suture tape, wire, string, cable, link, chain or other construct capable of carrying tension. A length of a suture of a cinch with one or more free ends or of a cinch loop may be from about 3 mm to 40 mm, such as from 5 mm to 30 mm or 7 mm to 20 mm. A suture useful for the cinches described herein includes a #5 or #6 suture and may be made from nylon, polyester, polyvinylidene fluoride (PVDF), or polypropylene or another biodegradable or non-biodegradable material.

The devices described herein may be configured for use in a bone or joint in the body and may cross one or more bones and/or connective tissues (e.g., one, two, three, four, five, etc. bones, ligaments, and/or tendons). In some variations, the implantable compression device 602 may have a maximum cross sectional size of between about 0.5 mm to 10 mm or anything in-between these values such as from 1 mm to 10 mm or from 2 mm to 8 mm. The maximum cross sectional size may refer to a diameter of a dynamic compression component, an anchor (either before or after insertion and expansion), a button or another component. In some implementations, the implantable compression devices disclosed herein remain implanted permanently. In some implementations, the devices are configured to be removable.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present disclosure.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the disclosure as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the disclosure as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. An implantable device comprising:
a first bone anchor configured to anchor in or on a first bone segment;
a second bone anchor configured to anchor in or on a second bone segment;
at least one suture interconnecting the first bone anchor and the second bone anchor;
a force-generating component connected to the at least one suture and configured to impart a force thereto,
wherein the first bone anchor, the second bone anchor, the at least one suture and the force-generating component are configured to cooperate together to draw the first bone segment and the second bone segment toward one another with a force in a predetermined range.

2. The implantable device of claim 1, wherein the force-generating component comprises a superelastic material, and wherein at least a portion of the predetermined range of force falls within a plateau region of a stress and strain curve of the superelastic material.

3. The implantable device of claim 2, wherein the predetermined range of force falls entirely within the plateau region, thereby causing the implantable device to impart a substantially constant force between the first bone segment and the second bone segment even when there is a change in a distance between the bone segments or between the bone anchors.

4. The implantable device of claim 3, wherein the superelastic material comprises nitinol.

5. The implantable device of claim 1, wherein the force-generating component is interconnected with the second bone anchor by the at least one suture, and wherein the force-generating component is interconnected with the first bone anchor by a separate second suture.

6. The implantable device of claim 1, wherein the force-generating component is interconnected with the second bone anchor by the at least one suture, and wherein the force-generating component is directly connected to the first bone anchor without a suture.

7. The implantable device of claim 6, wherein the force-generating component and the first bone anchor are integrally formed.

8. The implantable device of claim 7, wherein the integrally formed force-generating component and first bone anchor comprise a superelastic material.

9. The implantable device of claim 8, wherein the superelastic material comprises nitinol.

10. The implantable device of claim 1, wherein the force-generating component comprises a tube having a helical slit through a wall thickness thereof.

11. The implantable device of claim 1, wherein the force-generating component is directly connected to the at least one suture.

12. The implantable device of claim 1, wherein the implantable device further comprises a release tether configured to release a force drawing the first bone segment and the second bone segment toward one another.

13. The implantable device of claim 12, wherein the implantable device further comprises a pair of rings located between the first bone anchor and the second bone anchor, wherein the release tether is connected to one of the pair of rings, wherein the pair of rings is configured to alternately retain the force drawing the first bone segment and the second bone segment toward one another when there is no tension on the release tether, and release the force when there is a tension on the release tether.

14. A method of securing bone segments together comprising:
forming a channel through a first bone segment and at least partially through a second bone segment;
providing an implantable device having a first bone anchor, a second bone anchor, at least one suture interconnecting the first bone anchor and the second bone anchor, and a force-generating component connected to the at least one suture;
introducing the second bone anchor through the channel in the first bone segment and at least partially into the channel in the second bone segment;
introducing the force-generating component at least partially into the channel in the first bone segment;
locating the first bone anchor in or over a proximal opening in the channel through the first bone segment;
drawing the at least one suture in a proximal direction to cause the first bone anchor and the second bone anchor to be drawn toward one another with a force in a predetermined range.

15. The method of claim 14, wherein the second bone anchor is introduced past two cortical regions of the first bone segment and only one cortical region of the second bone segment, and wherein the second bone anchor is anchored in a cancellous bone region of the second bone segment.

16. The method of claim 14, wherein the second bone anchor is introduced past two cortical regions of the first bone segment and past two cortical regions of the second bone segment, and wherein the second bone anchor is anchored in a distal cortical bone region of the second bone segment.

17. The method of claim 14, wherein the first bone segment and the second bone segment are interconnected by a syndesmosis.

18. The method of claim 17, wherein the syndesmosis is a distal tibiofibular joint.

19. The method of claim 18, wherein the first bone segment is a distal fibula and the second bone segment is a distal tibia.

20. The method of claim 14, wherein the force-generating component comprises a superelastic material, and wherein at least a portion of the predetermined range of force falls within a plateau region of a stress and strain curve of the superelastic material.

21. The method of claim 20, wherein the predetermined range of force falls entirely within the plateau region, thereby causing the implantable device to impart a substantially constant force between the first bone segment and the second bone segment even when there is a change in a distance between the bone segments or between the bone anchors.

22. The method of claim 21, wherein the superelastic material comprises nitinol.

23. The method of claim 14, wherein the force-generating component is interconnected with the second bone anchor by the at least one suture, and wherein the force-generating component is interconnected with the first bone anchor by a separate second suture.

24. The method of claim 14, wherein the force-generating component is interconnected with the second bone anchor by the at least one suture, and wherein the force-generating component is directly connected to the first bone anchor without a suture.

25. The method of claim 24, wherein the force-generating component and the first bone anchor are integrally formed.

26. The method of claim 25, wherein the integrally formed force-generating component and first bone anchor comprise a superelastic material.

27. The method of claim 26, wherein the superelastic material comprises nitinol.

28. The method of claim 14, wherein the force-generating component comprises a tube having a helical slit through a wall thickness thereof.

29. The method of claim 28, wherein the method further comprises drawing the at least one suture in the proximal direction to cause the helical slit to widen until the first bone anchor and the second bone anchor are drawn toward one another with the force in the predetermined range.

30. The method of claim 14, wherein the force-generating component is directly connected to the at least one suture.

31. The method of claim 14, wherein the method further comprises drawing on a release tether of the implantable device to release the force drawing the first bone segment and the second bone segment toward one another.

32. The method of claim 30, wherein the implantable device further comprises a pair of rings located between the first bone anchor and the second bone anchor, wherein the release tether is connected to one of the pair of rings, wherein the pair of rings alternately retains the force drawing the first bone segment and the second bone segment toward one another when there is no tension on the release tether, and releases the force when there is a tension on the release tether.

33. An implantable device comprising:
a first bone anchor configured to anchor in or on a first bone segment;
a second bone anchor configured to anchor in or on a second bone segment; and
a force-generating component connected between the first bone anchor and the second bone anchor and configured to impart a force thereto, wherein the force-generating component comprises a tube having a helical slit through a wall thickness thereof, wherein the helical slit has a first end and a second end, wherein the first end comprises a tail extension which includes a curved portion and a straight portion, wherein the straight portion generally aligns with a longitudinal axis of the implantable device, and the curved portion transitions a trajectory of the helical slit between a normal pitch to a direction of the straight portion,
wherein the first bone anchor, the second bone anchor, and the force-generating component are configured to cooperate together to draw the first bone segment and the second bone segment toward one another with a force in a predetermined range.

34. The implantable device of claim 33, wherein the second end of the helical slit comprises a tail extension which includes a second curved portion and a second straight portion, wherein the second straight portion generally aligns with the longitudinal axis of the implantable device, and the second curved portion transitions a trajectory of the helical slit between a normal pitch to a direction of the second straight portion.

35. The implantable device of claim 33, wherein the device further comprises at least one suture interconnecting the force-generating component and the second bone anchor.

36. The implantable device of claim 33, wherein the force-generating component and the first bone anchor are integrally formed.

37. The implantable device of claim 36, wherein the integrally formed force-generating component and first bone anchor comprise a superelastic material.
